(19) United States
(12) Patent Application Publication
(10) Pub. No.: US 2022/0184173 A1
(43) Pub. Date: Jun. 16, 2022

(54) STABLE PHARMACEUTICAL FORMULATIONS OF PEPTIDE AND PROTEIN DRUGS

(71) Applicant: Emphascience, Inc., Pittsford, NY (US)

(72) Inventors: Rebanta Bandyopadhyay, Webster, NY (US); Susen Bandyopadhyay, Webster, NY (US); Meghan Rodriguez, Farmington, NY (US); Leo J. Magee, Stafford, NY (US); Leann J. Valentino, Rochester, NY (US)

(21) Appl. No.: 17/436,284

(22) PCT Filed: Mar. 5, 2020
(86) PCT No.: PCT/US2020/021204

§ 371 Date: Sep. 3, 2021

Related U.S. Application Data
(60) Provisional application No. 62/815,458, filed on Mar. 8, 2019.
(60) Provisional application No. 62/815,470, filed on Mar. 8, 2019.

(51) Int. Cl. A61K 38/14; A61K 47/10; A61K 38/12; A61K 38/13

(52) U.S. Cl. CPC A61K 38/14; A61K 38/13; A61K 38/12; A61K 47/10

(57) ABSTRACT

The present invention relates to compositions comprising a cyclicpeptide, a solvent and two or more hydroxylhydrocarbons, independently selected from i) ethanol and glycerol; or ii) ethanol and mannitol. Methods of treating diseased conditions using the compositions of the present invention are also provided in the present invention.

### FIELD OF THE INVENTION

The present invention is directed to compositions comprising a cyclicpeptide, a solvent and two or more hydroxylhydrocarbons, independently selected from i) ethanol and glycerol; or ii) ethanol and mannitol. The present invention is also directed to methods of treating diseased conditions using the compositions of the present invention.

### BACKGROUND OF THE INVENTION

All of about 7000 naturally occurring proteins and peptides identified so far, are known to play various critical roles in human physiology as hormones, neurotransmitters, growth factors, ion-channel ligands, and anti-infectives. Hence, proteins or peptides are active ingredients in more than 60 approved drug products in the United States alone. Several hundred proteins and peptides, both natural and modified are also under clinical and preclinical development as seen in Kaspar, A. A, “Future directions for peptide therapeutics” Drug Discovery Today, 18, 807-817 (2013); Hoffmann, K. F. “Peptide therapeutics: current status and future directions” Drug Discovery Today, 20(1), 122-128 (2015); and Dunn, J. L. “Therapeutic peptides: Historical perspectives, current development trends and future directions” Bioorganic and Medicinal Chemistry, 26, 2700-2707 (2018). Monoclonal antibodies are also growing as a new class of therapeutic proteins aimed at treating various types of cancer, inflammation, immune disorders and neurodegenerative diseases as seen, for example in Ecker, D. L, Jones, S. D. and Levin, H. L. “The therapeutic monoclonal antibody market” mAbs, 7(1):9-14 (2015).

However, proteins and peptides can be challenging to work with as they are often unstable in solution and can undergo hydrolysis, deamidation, oxidation, isomerization, or form aggregates that result in loss of pharmacological activity and reduce shelf-life of the drug product. For most proteins and peptides, factors such as pH and temperature of the solution, ionic strength, presence of impurities that can catalyze reactions, polarity, and viscosity may have an influence on the overall rates of deamidation.

The tricyclic glycopeptide antibiotic vancomycin, for example, was approved in the early 1950s for use against acute gram-positive bacterial infections and in many cases is the last line of defence in the treatment of infections caused by multi-drug-resistant bacteria. Due to susceptibility to decomposition, vancomycin as its hydrochloride salt is commonly available as a freeze-dried (lyophilized) powder. However, the lyophilized powder must be reconstituted before administration by intravenous infusion at the point of use and can only be used for 14 days when stored under refrigeration following reconstitution to a 50 mg/mL solution (Vancocin Package Insert). As the lyophilized powder form cannot be immediately used and requires aseptic reconstitution, as well as transfer and handling, the sterility and safety of the product can be compromised. This may add time, cost and inconvenience to the final user. A further commercially available form of vancomycin is a premixed solution of a ready to use vancomycin injection, USP in GALAXY Plastic Container (PL 2040) (NDA 050671), which is particularly susceptible to deamidation and, in order to reduce the rate of degradation, requires special storage conditions. The ready-to-use (RTU) and premixed vancomycin injection requires to be frozen and stored at a temperature of −20° C. (−4° F.) or below and prior to use, the frozen solution needs to be thawed and used within 72 hours when stored at room temperature (25° C./77° F.) or within 30 days when stored under refrigeration (5° C./41° F.). These requirements of gradual thawing and very low, sub-ambient storage temperature can also prevent ready availability and poses obstacles at the point of use.

Due to the drawbacks discussed above, many attempts have been made to improve the stability of pharmacologically active proteins and peptides in solution.

US 2014/0260098 A1, for example, discloses vancomycin hydrochloride solutions comprising trehalose and Tween (polyoxyethylene sorbitan esters). However, micellar colloidal solutions containing Tween or similar surface-active agents can alter the distribution of the drug in the human body following administration due to the changes in membrane permeability and partitioning characteristics of the drug-containing micelles. A reduced proportion of free, unsolubilized drug and loss of pharmacological efficacy may also result from such solutions.

Further examples of vancomycin solutions are disclosed in: WO 97/19690, which further comprise 0.5-30% (v/v) ethanol; WO 2016/071495 A1, which further comprise an amino acid or amino acid derivative such as N-acetyl-Glycine or N-acetyl-D-Alanine; U.S. Pat. No. 4,670,258, which further comprise acetylated dipeptides or tripeptides; and JP-1 1080021 and WO 2014/085526, which both further comprise amino acids or derivatives thereof.

Drawbacks of these approaches include the addition of inactive ingredients that have not previously been approved for pharmaceutical use or are considered to be safe only for limited patient types, manufacturing challenges due to loss of the added ingredients due to volatility or chemical instability, low solution concentration of the active that add to the dose volume and prevent dosing flexibility by all routes of administration. In addition, most of the known formulations do not provide a means for rapid and direct administration to eliminate the need for aseptic transfer, which would otherwise increase the risk of loss of sterility for sterile injectables. Also, when vancomycin, for example, is bound to an excipient that could compete with the binding of the drug to, for example, the bacterial cell wall, the proportion of free, unbound drug that is available to bind to the bacterial cell wall to exert its antibacterial effects may be reduced resulting in a loss of therapeutic effectiveness.

Therefore, there remains the need for further solutions of pharmacologically active proteins and/or peptides that address the issue of safety in a wide range of patient groups, and the stability of the pharmacologically active proteins or peptides. Also, there is a need for such solutions that are in readily usable dosage forms for various routes of administration and disease targets.

### SUMMARY OF THE INVENTION

The present invention is defined in the appended claims.

In accordance with a first aspect, the present invention provides a pharmaceutical composition comprising a cyclicpeptide, a solvent and two or more hydroxylhydrocarbons, wherein the two or more hydroxylhydrocarbons comprise i) ethanol and glycerol; or ii) ethanol and mannitol. Such compositions have been found to exhibit good stability of the cyclicpeptide in solution at room temperature and/or at ambient storage conditions.

Therefore, the present invention may provide advantages such as ease of storage, ease of handling, ease of manufacturing, ease of dilution and/or ease of administration. For example, in certain cases, by increasing the concentration of the pharmacologically active protein and/or peptide, the therapeutic dose can be administered by alternative routes without the need for further dilution. As a further example, the product defined by the present invention may be readily administered without thawing, in-situ compounding and/or aseptic transfers between more than one container. It is well known to those trained in the art of formulation and pharmaceutical product development that a ready to dilute solution can be packaged in juxtaposed chambers within a unit dosage form that can be mixed and diluted without the need for aseptic transfer. In some examples, additional pH adjustments are not required.

In accordance with a second aspect, there is provided a composition according to the first aspect for use in the treatment of a diseased condition.

The details, examples and preferences provided in relation to any particular one or more of the stated aspects of the present invention apply equally to all aspects of the present invention. Any combination of the embodiments, examples and preferences described herein in all possible variations thereof is encompassed by the present invention unless otherwise indicated herein, or otherwise clearly contradicted by context.

### BRIEF DESCRIPTION OF THE DRAWINGS

The invention will further be illustrated by reference to the following FIGURE:

FIG. 1: Temperature versus degradation rate of vancomycin composition according to Example 1.

It is understood that the following description and references to the FIGURES concern exemplary embodiments of the present invention and shall not be limiting the scope of the claims.

### DETAILED DESCRIPTION

The present invention is based on the surprising finding that adding two or more hydroxylhydrocarbons to a composition comprising a pharmacologically active protein and/or peptide and a solvent enhances the stability and retains adequate pharmacological activity of the pharmacologically active protein and/or peptide. In certain embodiments suitable stability was achieved. For example, the composition was stable at ambient conditions of storage for prolonged periods of time. Without wishing to be bound by theory, it is considered that in one mechanism, the presence of at least two hydroxylhydrocarbons in the present compositions reduces the overall rate of deamidation, i.e. breakage of one or more amide bonds by the effects of (i) lowering the free energy at the interface between the solvent and protein/peptide; and/or (ii) increasing the activation energy needed for such deamidation reaction. Without wishing to be bound by theory, it is hypothesized that the presence of the hydroxylhydrocarbons in sufficient quantities in the solution can disrupt the network of oriented, water-to-water molecule hydrogen (H—) bonded structures at the protein or peptide molecule and solvent interface, thereby reducing the interfacial free energy. In the present invention, the use of two or more hydroxylhydrocarbons selected from mono, di or polyhydroxylhydrocarbons are considered to lower the propensity of H-bonded networks among each of these hydroxylhydrocarbons and/or the water molecules, and thus reduce the interfacial free energy between protein or peptide and the solvent. Without wishing to be bound by theory, it is also considered that the method of improving the stability according to the invention may be applicable to all pharmacologically active peptides and/or proteins, especially those containing at least one asparagine or a glutamine residue or their corresponding acids. Examples of such pharmacologically active proteins and peptides also demonstrate surface hydrophobicity and are, in some cases, prone to form noncovalent dimers and/or higher order molecular associations in a polar medium.

### Pharmacologically Active Proteins and Peptides

Pharmaceutically active proteins or disclosed herein refer to any number of amino acids bonded to each other through amide bonds that demonstrate pharmacological activity when administered to human beings or animals in suitable quantities, as is the case for proteins or peptides that are biologically active. As disclosed herein, peptides have up to 50 amino acid residues and proteins have 50 or more amino acid residues. In some examples, the pharmaceutically active proteins and/or peptides contain at least one residue selected from asparagine, glutamine, aspartic amino acids and glutamic amino acids, as part of the primary sequence. In some examples the pharmaceutically active proteins and/or peptides have at least a secondary or a higher order structure/conformation. In some examples, the pharmacologically active proteins and/or peptides may also be chemically bonded to other types of chemical structures, such as, various types of sugars as in glycopeptides; lipids, as in lipopeptides; or other chemical entities that may be naturally found or may have been added to the indigenous protein or peptide for the purpose of modifying some property of such protein or peptide. In most cases, altering the conformation has a direct impact on the pharmacological activity. Examples of such pharmacologically active proteins and peptides include those that demonstrate surface hydrophobicity and are prone to form noncovalent dimers and higher order molecular associations in a polar medium.

In some examples, the pharmacologically active protein and/or peptide comprises at least a secondary or higher order structure and optionally an intramolecular bond forming a cyclic ring, such as vancomycin. In some examples, the pharmacologically active protein and/or peptide is selected from a glycopeptide, or a monoclonal antibody, a lipopeptide, pharmaceutically acceptable salts thereof, pharmaceutically acceptable solvates thereof, pharmaceutically acceptable hydrates thereof, or pharmaceutically acceptable co-crystals thereof.

In some examples the pharmacologically active protein and/or peptide comprises at least one residue selected from asparagine, glutamine, aspartic acid and/or glutamic acid residues. In some examples the pharmacologically active protein and/or peptide comprises at least one amide bond involving an asparagine, glutamine, aspartic acid and glutamic acid residue that is susceptible to deamidation via the formation of a cyclic imide intermediate. Such deamidation may particularly occur in a polar solvent.

In some examples, the pharmacologically active protein and/or peptide is selected from abaloparatide, abciximab, actaplanin, adalimumab, albiglutide, alemtuzumab, alirocumab, anidulafungin, atezolizumab, atosiban, avelumab, bacitracin, basiliximab, belimumab, benralizumab, bevacizumab, bezlotoxumab, blinatumomab, brodalumab, calcitonin, calcitonin-salmon, canakinumab, carbetocin, caspofungin, cetuximab, cibacalcin, colistin A and B, complestatin, cyclosporin, daclizumab, dalbavancin, daptomycin, daratumumab, denosumab, desirudin, desmethylvancomycin, desmopressin, dinutuximab, dulaglutide, dupilumab, durvalumab, eculizumab, elotuzumab, emicizumab, eptifibatide, evolocumab, exenatide, golimumab, guselkumab, idarucizumab, Infliximab, insulin, insulin aspart, insulin degludec, insulin detemir, insulin glargine, insulin glulisine, insulin lispro, insulin recombinant, ipilimumab, ixekizumab, lepirudin, linaclotide, liraglutide, lysine vasopressin, mepolizumab, micafungin, murepavadin, natalizumab, necitumumab, nesiritide, nivolumab, obiltoxaximab, obinutuzumab, ocrelizumab, ofatumumab, olaratumab, omalizumab, oritavancin, oxytocin, palivizumab, panitumumab, pembrolizumab, pertuzumab, plecanatide, pramlintide, ramucirumab, ranibizumab, raxibacumab, reslizumab, ristocetin, rituximab, sarilumab, secukinumab, semaglutide, siltuximab, teduglutide, teicoplanin, telavancin, tocilizumab, trastuzumab, trastuzumab-DKST, trastuzumab-DTTB, trastuzumab-PKRB, ustekinumab, vancomycin, vasoactive intestinal peptides, vasopressin, vedolizumab, ziconotide, β-avoparcin, pharmaceutical salts thereof, stereoisomers thereof, tautomers thereof, or derivatives thereof.

In certain embodiments pharmacologically active peptide is a cyclicpeptide selected from actaplanin, anidulafungin, atosiban, bacitracin, carbetocin, caspofungin, calcitonin, calcitonin-salmon, cibacalcin, colistin A and B, complestatin, cyclosporin, dalbavancin, daptomycin, desirudin, desmethylvancomycin, desmopressin, dulaglutide, eptifibatide, insulin, insulin aspart, insulin degludec, insulin detemir, insulin glargine, insulin glulisine, insulin lispro, insulin recombinant, lepirudin, linaclotide, lysine vasopressin, micafungin, murepavadin, nesiritide, oritavancin, oxytocin, plecanatide, pramlintide, ristocetin, teicoplanin, telavancin, vancomycin, vasopressin, ziconotide, β-avoparcin

In certain embodiments, the pharmacologically active peptide is vancomycin, pharmaceutical salts thereof, stereoisomers thereof, tautomers thereof, or derivatives thereof.

In certain embodiments, the pharmacologically active protein is present in an amount of about 0.01 to about 50 weight percent based on the total weight of the composition. For example, the pharmacologically active protein and peptide are present in an amount of at least 0.05, or at least 0.1, or at least 0.5, or at least 1.0, or at least 2.0, or at least 5.0, or at least 10.0 or at least 15.0 or at least 20.0 weight percent based on the total weight of the composition. For example, the pharmacologically active protein and peptide are present in an amount of at least 45, or at least 40, or at least 35, or at least 30, or at least 20, or at least 15 weight percent based on the total weight of the composition. For example, the pharmacologically active protein and peptide are present in an amount of about 1 to about 15 weight percent based on the total weight of the composition.

As used herein “composition” means “pharmaceutical composition”.

### Hydroxylhydrocarbons

The hydroxylhydrocarbons disclosed herein comprise a linear or cyclic hydrocarbon (CH) backbone or a combination thereof containing at least one hydroxyl (—OH) group attached to any one carbon atom therein. The hydroxylhydrocarbons may be aromatic or aliphatic and may contain heteroatoms. Optionally hydroxylhydrocarbons may contain other functional groups in addition to the hydroxyl (—OH) group, such as carboxylic acids, amino acids, esters, ethers, amines, sugars, alcohols and should at least comprise one or more free (unbounded) hydroxyl (—OH) substituents. The hydroxylhydrocarbons of the present invention are generally recognized as safe (GRAS), suitable for pharmaceutical use and human administration.

The hydroxylhydrocarbons may be independently selected from monohydroxylhydrocarbons, dihydroxylhydrocarbons, and polyhydroxylhydrocarbons. In certain embodiments, there is no more than one hydroxylhydrocarbon selected from each of the groups monohydroxylhydrocarbons, dihydroxylhydrocarbons, and polyhydroxylhydrocarbons. For example, the hydroxylhydrocarbons may be one monohydroxylhydrocarbon and one dihydroxylhydrocarbon, or one monohydroxylhydrocarbon and one polyhydroxylhydrocarbon, or one dihydroxylhydrocarbon and one polyhydroxylhydrocarbon.

Monohydroxylhydrocarbons disclosed herein comprise a hydrocarbon backbone substituted with one hydroxyl (—OH) group bonded to a carbon atom. Dihydroxylhydrocarbons disclosed herein comprise a hydrocarbon backbone substituted with two hydroxyl groups. Polyhydroxylhydrocarbons disclosed herein comprise a hydrocarbon backbone substituted with three or more hydroxyl groups.

Examples of pharmaceutically acceptable monohydroxylhydrocarbons include, but are not limited to, ethanol, ethanol and water fixed composition mixtures commonly referred to as alcohol, benzyl alcohol, Alitame, alpha-tocopherol, aspartame, benzoic acid, calcium lactate, cetostearyl alcohol, chlorobutanol, chlorocresol, chloroxylenol, cholesterol, cresol, diethyleneglycol monoethylether, ethyl lactate, ethyl maltol, ethyl vanillin, glycofurol, hydroxyethylpiperazine ethane sulfonic acid (HEPES), lauric acid, maltol, menthol, methionine, monoethanolamine, monosodium glutamate, neotame, phenol, phenoxyethanol, phenylethyl alcohol, phenylmercuric borate, polyoxyethylene alkyl ethers, polyoxyethylene monostearate, propionic acid, propylene glycol monolaurate, propyl paraben, sodium formaldehyde sulfoxylate, sodium lactate, sorbic acid, stearic acid, stearyl alcohol, thymol and vitamin E polyethylene glycol succinate.

Examples of dihydroxylhydrocarbons include, but are not limited to, propylene glycol, polyethylene glycol, lactic acid, adipic acid, aluminium monostearate, bronopol, butylene glycol, diethanolamine, dipropylene glycol, disodium edetate, fumaric acid, imidurea, maleic acid, monothioglycerol, phenylmercuric orthoborate, poloxamer, poly(DL-lactic acid), polyethylene oxide and vanillin.

Examples of polyhydroxylhydrocarbons include, but are not limited to, glycerol, mannitol, sorbitol, maltitol, xylitol, meglumine, trehalose, sucrose, sucralose, sucrose palmitate, sucrose stearate, tartaric acid, sorbitan and polyoxyethylated sorbitan esters, acacia, acetic acid, acetone sulfite, alginic acid and its esters and salts, sodium, potassium, ammonium and calcium alginate for example, ascorbic acid, ascorbyl palmitate, boric acid, carboxymethylcellulose sodium, carrageenan, castor oil, hydrogenated castor oil, polyoxyethylated castor oil derivatives, citric acid, corn syrup, cyclodextrins of different sizes (alpha, beta, or gamma), dextran, dextrates, dextrin, dextrose, edetic acid, erythorbic acid, erythritol, ethylene glycol and vinyl alcohol grafted copolymer, fructose, galactose, gelatin, glucose, hydroxyethyl cellulose, hydroxyethyl methylcellulose, derivatized cyclodextrins, such as hydroxypropyl β-cyclodextrin and sulfobutylether β-cyclodextrin, hydroxypropyl cellulose, hydroxypropyl methylcellulose (Hypromellose), inulin, isomalt, lactitol, lactose, malic acid, maltodextrin, maltose, D-mannose, pectin, pentetic acid, polycarbophil, polydextrose, polymethacrylates, polyvinyl acetate phthalate, polyvinyl alcohol, propyl gallate, pullulan, raffinose, sodium ascorbate, sodium hyaluronate, pregelatinized starch, tagatose, tartaric acid, triethanolamine, tromethamine, xanthan and zein.

In some examples the pharmaceutical composition comprises a monohydroxylhydrocarbon and a polyhydroxylhydrocarbon. In some examples, the monohydroxylhydrocarbon is ethanol. In some examples, the polyhydroxylhydrocarbon is glycerol. In some examples the composition comprises ethanol and glycerol. In some examples the composition comprises ethanol as the monohydroxylhydrocarbon and mannitol as a polyhydroxylhydrocarbon. In some examples, the dihydroxylhydrocarbon is polyethylene glycol and a polyhydroxylhydrocarbon is glycerol. In some examples, the polyhydroxylhydrocarbon is glycerol and the dihydroxylhydrocarbon is propylene glycol. In some examples, the composition comprises ethanol and sucrose, ethanol and mannitol, ethanol and lactose, ethanol and dextrose, ethanol and cyclodextrin, ethanol and sucralose, or ethanol and sorbitol.

In certain embodiments the composition comprises vancomycin or pharmaceutically acceptable salts thereof, water, glycerol and ethanol. In certain embodiments the composition comprises vancomycin or pharmaceutically acceptable salts thereof, water, mannitol and ethanol.

In some examples the two or more hydroxylhydrocarbons are present in the composition in an amount from about 1.0 to about 60 weight percent based on the total weight of the composition. For example, the two or more hydroxylhydrocarbons are present in the composition in an amount from about 1.05 to about 58, or from about 1.1 to about 56, or from about 1.2 to about 56, or from about 1.5 to about 54, or from about 1.8 to about 52, or from about 2 to about 50, or from about 2.5 to about 48, or from about 3 to about 46, or from about 4 to about 44, or from about 6 to about 42, or from about 8 to about 40, or from about 10 to about 38, or from about 12 to about 36, or from about 14 to about 34, or from about 16 to about 32, or from about 18 to about 30, or from about 20 to about 28, or from about 22 to about 26 weight percent based on the total weight of the composition.

In certain embodiments, ethanol is present in the composition in an amount of from about 1 to about 40 weight percent based on the total weight of the composition. For example, glycerol is present in the composition in an amount of from about 2 to about 38, or from about 3 to about 36, or from about 4 to about 34, or from about 5 to about 32, or from about 6 to about 30, or from about 7 to about 28, or from about 8 to about 26, or from about 9 to about 24, or from about 10 to about 22, or from about 11 to about 20, or from about 12 to about 18, or from about 14 to about 16 weight percent based on the total weight of the composition.

In certain embodiments, glycerol is present in the composition in an amount of from about 1 to about 22 weight percent based on the total weight of the composition. For example, glycerol is present in the composition in an amount of from about 2 to about 21, or from about 3 to about 20, or from about 4 to about 19, or from about 5 to about 18, or from about 6 to about 17, or from about 7 to about 16, or from about 8 to about 15, or from about 9 to about 14, or from about 10 to about 13, or from about 11 to about 12 weight percent based on the total weight of the composition.

In certain embodiments, mannitol is present in the composition in an amount of about from about 1 to about 22 weight percent based on the total weight of the composition. For example, glycerol is present in the composition in an amount of from about 2 to about 21, or from about 3 to about 20, or from about 4 to about 19, or from about 5 to about 18, or from about 6 to about 17, or from about 7 to about 16, or from about 8 to about 15, or from about 9 to about 14, or from about 10 to about 13, or from about 11 to about 12 weight percent based on the total weight of the composition.

In some examples, each of the monohydroxylhydrocarbons, dihydroxylhydrocarbons and polyhydroxylhydrocarbons are independently present in the composition in an amount from about 0.75 to about 59.25 weight percent based on the total weight of the composition. For example, each of the monohydroxylhydrocarbons, dihydroxylhydrocarbons and polyhydroxylhydrocarbons are independently present in the composition in an amount from about 0.80 to about 59, or from about 0.90 to about 57, or from about 1.0 to about 55, or from about 1.5 to about 54, or from about 1.8 to about 52, or from about 2 to about 50, or from about 2.5 to about 48, or from about 3 to about 46, or from about 4 to about 44, or from about 6 to about 42, or from about 8 to about 40, or from about 10 to about 38, or from about 12 to about 36, or from about 14 to about 34, or from about 16 to about 32, or from about 18 to about 30, or from about 20 to about 28, or from about 22 to about 26 weight percent based on the total weight of the composition.

In certain embodiments the pH of the composition is between 2.5 and 11, or between 4 and 11, or between 3 and 10, or between 4 and 9, or between 5 and 8, or between 6 and 7, or between 3 and 5, or between 3.5 and 4. The optimum pH may be defined by the pH of the solution that sets the ground state and highest activation energy for any change of conformation associated with deamidation. In some examples, the final pH of the solution composition may be altered to match the optimum range using standard solutions of either hydrochloric acid (for lowering pH) or sodium hydroxide (for raising the pH) according to common practice in the pharmaceutical field.

In certain embodiments the composition is substantially free from non-bonded amino acids, such as tryptophan and/or salts thereof, substantially free from N-acyl-D-alanine, N-acyl-glycine and/or salts thereof or substantially free from N-acetyl-D-alanine, N-acetyl-glycine or salts thereof. As used herein, “substantially free” means the total absence or near absence of a component. Trace amount may be present, but the skilled person would understand that substantially free is an amount that is equal to or lower than the acceptable limits set by the standard grade of materials suitable for the purpose of therapeutic use. For example, “substantially free” means that the said component is present in an amount of up to about 0.1 weight percent, or up to about 0.05 weight percent, based on the total weight of the composition.

In certain embodiments, the solvent used according to the invention is a polar solvent such as water, dimethylsulfoxide, dimethylacetamide, monoethanolamine, polyethylene glycol, propylene glycol or mixtures thereof. In certain embodiments the polar solvent used is water.

In certain embodiments, the water is of a quality that is suitable for pharmaceutical use and may include, but not limited to, purified water, sterile purified water, water for injection, sterile water for injection, bacteriostatic water for injection, sterile water for inhalation, sterile water for irrigation, drinking water, water for hemodialysis, distilled water, freshly distilled water, ammonia-free water, carbon-di-oxide-free water, deionized water, deionized distilled water, filtered water, high-purity water, deaerated water and/or oxygen-free water. In some embodiments, the water has a pH in the range of 4 to 9 with or without adjustment. In certain embodiments, solvent is water and the water has an initial pH of between 4.0 and 9.0, or between 4.5 and 8.0, or between 5.0 and 7.0, or between 5.5 and 6.5, or between 6.5 and 7.5, or between 5.0 and 5.5.

In certain embodiments, the solvent is present in the composition in an amount from about 40 to about 99 weight percent based on the total weight of the composition. For example, the solvent is present in the composition in an amount from about 42 to about 97, or from about 44 to about 95, or from about 46 to about 93, or from about 48 to about 91, or from about 46 to about 90, or from about 50 to about 85, or from about 55 to about 80, or from about 60 to about 75, or from about 55 to about 70, or from about 60 to about 65 weight percent based on the total weight of the composition.

In certain embodiments, the composition is a solution, a suspension, or a mixture thereof. As used herein, a suspension is a mixture of one or more distinct solid, or liquid phase components that are not completely miscible and retain their individual melting and/or boiling points. As used herein, a solution is defined a homogenous mixture where the melting and/or boiling points of the individual components are no longer apparent. In some examples, the individual components of the composition are stored separately and mixed directly before use.

In certain embodiments, the composition further comprises sweeteners selected from glucose, sucrose, sorbitol, xylitol, maltitol, sucralose, sodium saccharin, aspartame, stevia or another glycoside and mixtures thereof.

### Stability/Loss of Purity

The stability of the composition of the present invention may be monitored using a number of methods. The stability may be determined by establishing the initial amount of pharmaceutically active protein and peptide, and then measuring the amount of pharmaceutically active protein and peptide remaining after a certain time thereafter and comparing the two values. The initial amount of pharmaceutically active protein and peptide is the amount present immediately after mixing all the components of the composition. The amount of pharmaceutically active peptide present may be measured using a range of methods known in the art, such as HPLC, mass spectrometry, spectrophotometry, gel electrophoresis, Western Blotting, light scattering, microbiological or other biological activity measuring assays. A typical method of tracking protein and/or peptide stability would constitute comparing the purity of the protein or peptide in a given product formulation against that of a freshly prepared standard to calculate the amount of undenatured, nondegraded, or native protein or peptide in the product for any given sample. Samples that are stored and analyzed over various periods of time would then provide a quantitative profile of the purity of the protein or peptide over time. Optionally, the degradation rate of the protein or peptide under stressed conditions of storage, such as at an elevated temperature, can then be determined from the decreasing purity versus time profile by fitting suitable regression lines or curves. Such degradation rates generated from stressed stability studies are particularly useful in comparing between different product formulations over a short period of time.

In certain embodiments, at least 90% by weight of the pharmacologically active protein and peptide is present in the composition after being stored for 30 days at from about 20 to about 25° C., based on the initial amount of the pharmacologically active protein and peptide. For example, at least 92%, or at least 94%, or at least 96%, or at least 98% of the pharmacologically active protein and peptide is present in the composition after being stored for 30 days at from about 20 to about 25° C., based on the initial amount of the pharmacologically active protein and peptide. For example, the composition is stored at about 25° C., or at about 24° C., or at about 23° C., or at about 22° C., or at about 21° C.

In certain embodiments, the at least 97% by weight of the pharmacologically active protein and peptide is present in the composition after being stored for 30 days at from about 2 to about 8° C., based on the initial amount of the pharmacologically active protein and peptide. For example, at least 97.5%, or at least 98%, or at least 98.5%, or at least 99%, at least 99.5% of the pharmacologically active protein and peptide is present in the composition after being stored for 30 days at from about 2 to about 8° C., based on the initial amount of the pharmacologically active protein and peptide. For example, the composition is stored at about 3° C., at about 4° C., at about 5° C. at about 6° C., at about 7° C.

As mentioned above, the purity of the composition according to the present invention may be monitored using one or more analytical methods from those listed before that are most suited for analyzing the protein or peptide in question. The loss in purity may be determined by subtracting the purity of the pharmaceutically active peptide in the product at any given time from that immediately after manufacturing of the product (time to). The difference in purities would constitute the loss of purity over the time period of testing. Alternatively, the purity of the protein or peptide could be measured at various time points from samples that are manufactured and stored in suitable sealed containers, which represent the unit dosage form. The purities are then plotted against time and fitted to a regression line, if linear, to determine an overall pseudo first-order degradation rate from the slope of such regression line.

In some examples, compositions disclosed herein are manufactured by mixing the pharmacologically active protein and/or peptide with two or more hydroxylhydrocarbons in a solvent. In one example, the components are mixed in a mixing vessel until completely dissolved and, then, filling the finished solution into type 1 glass vials sealed with rubber stopper and flip-top crimp-cap. The purity of the pharmaceutically active protein and/or peptide was determined by HPLC immediately after manufacturing by comparing to a standard solution of the active. The purity of the active was also analyzed from different vials after fixed time intervals and plotted against time to form a linear relationship. A pseudo first-order daily degradation rate was estimated from the slope of the regression line fitted to the purity versus time plot.

In certain embodiments, the purity of the pharmaceutically active protein and/or peptide may simply be determined from the relative (%) peak are of the active in a chromatogram obtained by HPLC as compared to that from other related substances and degradation products in the same chromatogram without the need for a standard solution. Such a strategy for determining the purity of the protein or peptide may be particularly applicable for analytical methods when the detection method is adequately sensitive to detect and quantify related substances produced from the deamidation of the protein or peptide and there is suitable mass balance as can be easily ascertained by those trained in the art of pharmaceutical analysis.

In certain embodiments the loss of purity of the pharmacologically active protein and peptide when stored between about 20 and about 25° C. is no more than 0.3% per day. For example, the loss of purity of the pharmacologically active protein and peptide when stored at from about 20 to about 25° C. is no more than 0.25% per day, or no more than 0.2% per day, or no more than 0.15% per day, or no more than 0.1% per day, or no more than 0.05% per day. For example, the composition is stored at about 25° C., or at about 24° C., or at about 23° C., or at about 22° C., or at about 21° C.

In certain embodiments the loss of purity of the pharmacologically active protein and peptide when stored at from about 2 to about 8° C. is no more than 0.03% per day. For example, the loss of purity of the pharmacologically active protein and peptide when stored between about 2 and about 8° C. is no more than 0.025% per day, or no more than 0.02% per day, or no more than 0.015% per day, or no more than 0.01% per day, or no more than 0.005% per day. For example, the composition is stored at about 3° C., at about 4° C., at about 5° C. at about 6° C., at about 7° C.

In some examples, a kit of the composition disclosed herein may be stored under refrigeration between about 2 and about 8° C. for up to 5 years before use, or up to 4.5 years, or up to 4 years, or up to 3.5 years or up to 3 years or up to 2.5 years or up to 2 years, or at least 1 year, or at least 1.5 years, or at least 2 years, or at least 2.5 years.

In certain embodiments composition according to the invention is a pharmaceutical composition.

In certain embodiments the compositions according to the invention are used for the treatment of a diseased condition. The diseased condition includes cancer, inflammatory diseases, diseases of the immune system, diseases of the nervous system, diseases of the circulatory systems, metabolic disorders, pain, diabetes, infections and combinations thereof.

In certain embodiments cancer may be skin cancer, bile duct cancer, bladder cancer, bone cancer, brain cancer, breast cancer, bronchial cancer, leukemia, lymphoma, carcinoid cancer, cervical cancer, chronic lymphocytic leukemia, chronic myelogenous leukemia, colorectal cancer, uterine cancer, head and neck cancer, extragonadal cancer, stomach cancer, kidney cancer, liver cancer, lung cancer, eye cancer, metastatic cancer, ovarian cancer, pancreatic cancer, parathyroid cancer, thyroid cancer, prostate cancer, rectal cancer, sarcoma, testicular cancer, urethral cancer, uterine cancer, vaginal cancer, vascular tumors, vulvar cancer. In certain embodiments, inflammatory diseases may be allergies, asthma, atherosclerosis, atopic dermatitis, autoinflammatory syndromes, Morbus Crohn, inflammatory bowel disease, psoriasis, ulcerative colitis, chronic peptic ulcer, rheumatoid arthritis, periodontitis, sinusitis, active hepatitis, lupus erythematodes, osteoporosis. In certain embodiments diseases of the immune system may be HIV, multiple sclerosis, Morbus Behcet syndrome, DiGeorge syndrome, selective immunglobuline A deficiency, Wiskott-Aldrich syndrome, diabetes type I, rheumatoid arthritis, allergies, rejection of organs, Morbus Crohn, osteoporosis, lupus erythematodes. In certain embodiments diseases of the nervous system may be stroke, aneurysma, Parkinson, multiple sclerosis, epilepsy, meningitis, cranio-cerebral trauma, migraine, polyneuropathy, brain tumor. In certain embodiments diseases of the circulatory systems may be coronary heart disease, angina, heart attack, congenital heart disease, hypertension, stroke, vascular dementia, heart valve disease, cardiomyopathy, artherosclerosis. In certain embodiments metabolic disorders may be metabolic syndrome, metabolic disorder or inborn errors of metabolism. In certain embodiments diabetes may be diabetes type I, diabetes type II, pregnancy-related diabetes, late autoimmune diabetes in adults, maturity-onset diabetes in the young, insulin resistance. In certain embodiments pain may be migraine, tension headache, cluster headache, neuralgia, back pain, pain due to rheumatoid arthritis, and pain from tumor. In certain embodiments, the infections may be bacterial, fungal and/or parasitic infection. Examples of such infections include, but are not limited to, infections due to gram-positive bacilli including anthrax, diphtheria, enterococcal infections, erysipelothricosis, listeriosis, those due to gram-positive cocci, including pneumococcal infections, staphylococcal aureus infections, staphylococcal endocarditis, Streptococcal infections, infections due to various Clostridium species, including, Clostridium difficile, C. botulinum, C. perfringens, C. tetani, C. sordellii, C. welchii, Toxic shock syndrome, infections of the stomach, upper and lower gastrointestinal tract, enterocolitis, infections of the kidney, bladder and the urethra, pneumonia, tuberculosis, rabies, septicemia, bone infections, lower respiratory tract infections, infections of the brain, meninges, subarachnoid space and surrounding structures, and skin and skin structure infections.

In certain embodiments compositions according to the invention can be administered directly into the skin or around the skin structure, into the blood stream, into muscle, into tissue, into fat, or into an internal organ, such as the brain or the lung. Suitable means for parenteral administration include intravenous, intraarterial, intraperitoneal, intrathecal, intraventricular, intraurethral, intrasternal, intracranial, intramuscular, intra-ossial, intradermal and subcutaneous. Suitable devices for parenteral administration include needle (including microneedle, microprojections, soluble needles and other micropore formation techniques) injectors, needle-free injectors and infusion techniques. The compositions according to the invention may also be formulated for oral administration such as liquid filled capsules, or spray dried, tray-dried or freeze-dried or sprayed on to a solid substrate to form solid phases that are then redissolved, or resuspended or compressed or molded into other dosage forms, such as, tablets, capsules, suppositories, inserts or patches.

For the avoidance of doubt, the present application is directed to subject-matter described in the following numbered paragraphs.
1. A pharmaceutical composition comprising a pharmacologically active protein and/or peptide, a solvent and two or more hydroxylhydrocarbons, wherein the two or more hydroxylhydrocarbons comprise i) ethanol and glycerol; or ii) ethanol and mannitol.
2. The composition according to numbered paragraph 1, wherein the pharmacologically active protein and/or peptide comprises at least a secondary or a higher order structure and, optionally, an intramolecular bond forming a cyclic ring.
3. The composition according to numbered paragraph 1 or numbered paragraph 2, wherein the pharmacologically active protein and/or peptide comprises at least one residue selected from asparagine, glutamine, aspartic acid and glutamic acid residues.
4. The composition according to any preceding numbered paragraph, wherein the pharmacologically active protein and/or peptide is selected from a glycopeptide, a lipopeptide, a monoclonal antibody, pharmaceutically acceptable salts or esters thereof, pharmaceutically acceptable solvates thereof, pharmaceutically acceptable hydrates thereof, or pharmaceutically acceptable co-crystals thereof.
5. The pharmaceutical composition according to any preceding numbered paragraph, wherein the pharmacologically active protein and/or peptide is selected from abaloparatide, abciximab, actaplanin, adalimumab, albiglutide, alemtuzumab, alirocumab, anidulafungin, atezolizumab, atosiban, avelumab, bacitracin, basiliximab, belimumab, benralizumab, bevacizumab, bezlotoxumab, blinatumomab, brodalumab, calcitonin, calcitonin-salmon, canakinumab, carbetocin, caspofungin, cetuximab, cibacalcin, colistin A and B, complestatin, cyclosporin, daclizumab, dalbavancin, daptomycin, daratumumab, denosumab, desirudin, desmethylvancomycin, desmopressin, dinutuximab, dulaglutide, dupilumab, durvalumab, eculizumab, elotuzumab, emicizumab. eptifibatide, evolocumab, exenatide, golimumab, guselkumab, idarucizumab, Infliximab, insulin, insulin aspart, insulin degludec, insulin detemir, insulin glargine, insulin glulisine, insulin lispro, insulin recombinant, ipilimumab, ixekizumab, lepirudin, linaclotide, liraglutide, lysine vasopressin, mepolizumab, micafungin, murepavadin, natalizumab, necitumumab, nesiritide, nivolumab, obiltoxaximab, obinutuzumab, ocrelizumab, ofatumumab, olaratumab, omalizumab, oritavancin, oxytocin, palivizumab, panitumumab, pembrolizumab, pertuzumab, plecanatide, pramlintide, ramucirumab, ranibizumab, raxibacumab, reslizumab, ristocetin, rituximab, sarilumab, secukinumab, semaglutide, siltuximab, teduglutide, teicoplanin, telavancin, tocilizumab, trastuzumab, trastuzumab-dkst, trastuzumab-dttb, trastuzumab-pkrb, ustekinumab, vancomycin, vasoactive intestinal peptides, vasopressin, vedolizumab, ziconotide, β-avoparcin, pharmaceutical salts thereof, stereoisomers thereof, tautomers thereof, or derivatives thereof.
6. The pharmaceutical composition according to any preceding numbered paragraph, wherein the pharmacologically active peptide is vancomycin, pharmaceutical salts thereof, stereoisomers thereof, tautomers thereof, or derivatives thereof.
7. The pharmaceutical composition according to any preceding numbered paragraph, wherein the composition is substantially free from non-bonded amino acids and/or salts thereof.
8. The pharmaceutical composition according to any preceding numbered paragraph, wherein the composition is substantially free from N-acyl-D-alanine, N-acyl-glycine and/or salts thereof, such as N-acetyl-D-alanine, N-acetyl-glycine or salts thereof.
9. The pharmaceutical composition according to any preceding numbered paragraph, wherein the pharmacologically active protein and peptide are present in an amount of about 0.01 to about 50 weight percent based on the total weight of the composition.
10. The pharmaceutical composition according any preceding numbered paragraph, wherein the composition is a solution, a suspension, or a mixture thereof.
11. The pharmaceutical composition according to any preceding numbered paragraph, wherein the solvent is a polar solvent, selected from water, dimethylsulfoxide, dimethylacetamide, monoethanolamine, polyethylene glycol, propylene glycol and mixtures thereof.
12. The pharmaceutical composition according to any preceding numbered paragraph, wherein the solvent is water with an initial pH between 5.0 and 5.5
13. The pharmaceutical composition according to any preceding numbered paragraph, wherein the total amount of hydroxylhydrocarbon is from about 1 to about 60 weight percent based on the total weight of the composition.
14. The pharmaceutical composition according to any preceding numbered paragraph, wherein the amount of ethanol is from about 1 to about 40 weight percent based on the total weight of the composition.
15. The pharmaceutical composition according to any preceding numbered paragraph, wherein the amount of glycerol or mannitol is from about 1 to about 22 weight percent based on the total weight of the composition.
16. The pharmaceutical composition according to any preceding numbered paragraph, further comprising sweeteners selected from glucose, sucrose, sorbitol, xylitol, maltitol, sucralose, sodium saccharin, aspartame, stevia or another glycoside and mixtures thereof.
17. The pharmaceutical composition according to any preceding numbered paragraph, wherein at least 90% by weight of the pharmacologically active protein and peptide is present in the composition after being stored for 30 days at between about 20 and about 25° C., based on the initial amount of the pharmacologically active protein and peptide.
18. The pharmaceutical composition according to any preceding numbered paragraph, wherein at least 97% by weight of the pharmacologically active protein and peptide is present in the composition after being stored for 30 days at between about 2 about and 8° C. based on the initial amount of the pharmacologically active protein and peptide.
19. The pharmaceutical composition according to any preceding numbered paragraph, wherein the loss of purity of the pharmacologically active protein and peptide when stored between about 20 and about 25° C. is no more than 0.3% per day.
20. The pharmaceutical composition according to any preceding numbered paragraph, wherein the loss of purity of the pharmacologically active protein and peptide when stored between about 2 and about 8° C. is no more than 0.03% per day.
21. A method of stabilizing a pharmaceutical composition comprising a pharmacologically active protein and/or peptide in a solvent by the addition of two or more hydroxylhydrocarbons, wherein the two or more hydroxylhydrocarbons comprise i) ethanol and glycerol; or ii) ethanol and mannitol.
22. The method according to numbered paragraph 21, wherein the pharmacologically active protein and/or peptide comprises at least a secondary or a higher order structure and, optionally, one intramolecular bond forming a cyclic ring.
23. The method according to any one of numbered paragraphs 21 to 22, wherein the pharmacologically active protein and/or peptide comprises at least one residue selected from asparagine, glutamine, aspartic acid and/or glutamic acid residues.
24. The method according to any one of numbered paragraphs 21 to 23, wherein the pharmacologically active protein and/or peptide is selected from a glycopeptide, a lipopeptide, a monoclonal antibody, pharmaceutically acceptable salts thereof, pharmaceutically acceptable solvates thereof, pharmaceutically acceptable hydrates thereof, or pharmaceutically acceptable co-crystals thereof.
25. The method according to any one of numbered paragraphs 21 to 24, wherein the pharmacologically active protein and/or peptide is selected from abaloparatide, abciximab, actaplanin, adalimumab, albiglutide, Alemtuzumab, alirocumab, anidulafungin, atezolizumab, atosiban, avelumab, bacitracin, basiliximab, belimumab, benralizumab, bevacizumab, bezlotoxumab, blinatumomab, brodalumab, calcitonin, calcitonin-salmon, canakinumab, carbetocin, caspofungin, cetuximab, cibacalcin, colistin A and B, complestatin, cyclosporin, daclizumab, dalbavancin, daptomycin, daratumumab, denosumab, desirudin, desmethylvancomycin, desmopressin, dinutuximab, dulaglutide, dupilumab, durvalumab, eculizumab, elotuzumab, emicizumab. eptifibatide, evolocumab, exenatide, golimumab, guselkumab, idarucizumab, Infliximab, insulin, insulin aspart, insulin degludec, insulin detemir, insulin glargine, insulin glulisine, insulin lispro, insulin recombinant, ipilimumab, ixekizumab, lepirudin, linaclotide, liraglutide, lysine vasopressin, mepolizumab, micafungin, murepavadin, natalizumab, necitumumab, nesiritide, nivolumab, obiltoxaximab, obinutuzumab, ocrelizumab, ofatumumab, olaratumab, omalizumab, oritavancin, oxytocin, palivizumab, panitumumab, pembrolizumab, pertuzumab, plecanatide, pramlintide, ramucirumab, ranibizumab, raxibacumab, reslizumab, ristocetin, rituximab, sarilumab, secukinumab, semaglutide, siltuximab, teduglutide, teicoplanin, telavancin, tocilizumab, trastuzumab, trastuzumab-dkst, trastuzumab-dttb, trastuzumab-pkrb, ustekinumab, vancomycin, vasoactive intestinal peptides, vasopressin, vedolizumab, ziconotide, β-avoparcin, pharmaceutical salts thereof, stereoisomers thereof, tautomers thereof, or derivatives thereof.
26. The method according to any one of numbered paragraphs 21 to 25, wherein the pharmacologically active peptide is vancomycin, pharmaceutical salts thereof, stereoisomers thereof, tautomers thereof, or derivatives thereof.
27. The method according to any one of numbered paragraphs 21 to 26, wherein the composition is substantially free from non-bonded amino acids and/or salts thereof.
28. The method according to any one of numbered paragraphs 21 to 27, wherein the composition is substantially free from N-acyl-D-alanine, N-acyl-glycine and/or salts thereof, such as N-acetyl-D-alanine, N-acetyl-glycine and/or salts thereof.
29. The method according to any one of numbered paragraphs 21 to 28, wherein the pharmacologically active protein and peptide are present in an amount of about 0.01 to about 50 weight percent based on the total weight of the composition.
30. The method according to any one of numbered paragraphs 21 to 29, wherein the composition is a solution, a suspension or a mixture thereof.
31. The method according to any one of numbered paragraphs 21 to 30, wherein the solvent is a polar solvent, selected from water, dimethylsulfoxide, dimethylacetamide, monoethanolamine, polyethylene glycol, propylene glycol and mixtures thereof.
32. The method according to any one of numbered paragraphs 21 to 31, wherein the solvent is water with an initial pH between 5.0 and 5.5.
33. The method according to any one of numbered paragraphs 21 to 32, wherein total amount of hydroxylhydrocarbon is present in an amount from about 1 to about 60 weight percent based on the total weight of the composition.
34. The method according to any one of numbered paragraphs 21 to 22, further comprising sweeteners selected from glucose, sucrose, sorbitol, xylitol, maltitol, sucralose, sodium saccharin, aspartame, stevia or another glycoside and mixtures thereof.
35. The method according to any one of numbered paragraphs 21 to 34, wherein at least 90% by weight of the pharmacologically active protein and peptide is present in the composition after being stored for 30 days at between about 20 and about 25° C., based on the initial amount of the pharmacologically active protein and peptide.
36. The method according to any one of numbered paragraphs 21 to 35, wherein at least 97% by weight of the pharmacologically active protein and peptide is present in the composition after being stored for 30 days between about 2 and about 8° C., based on the initial amount of the pharmacologically active protein and peptide.
37. The method according to any one of numbered paragraphs 21 to 36, wherein the loss of purity of the pharmacologically active protein and peptide when stored between about 22 and about 25° C. is no more than 0.3% per day.
38. The method according to any one of numbered paragraphs 21 to 37, wherein the loss of purity of the pharmacologically active protein and peptide when stored between about 2 and about 8° C. is no more than 0.03% per day.
39. A pharmaceutical composition obtained by any one of the methods of numbered paragraphs 21 to 38.
40. A method for the treatment of a diseased condition comprising the step of administering a pharmaceutically effective amount of the composition according to any one of numbered paragraphs 1 to 20 or numbered paragraph 39 to a subject in need thereof.
41. A method of numbered paragraph 40, wherein the diseased condition is selected from cancer, inflammatory diseases, diseases of the immune system, diseases of the nervous system, diseases of the circulatory systems, metabolic disorders, pain, diabetes, infections and combinations thereof.
42. A method of numbered paragraph 41, wherein the infection is a bacterial infection, a fungal infection and/or a parasitic infection.
43. A pharmaceutical composition according to any one of numbered paragraphs 1 to 20 or numbered paragraph 39 for use in a diseased condition.
44. The composition for use according to numbered paragraph 43, wherein the diseased condition is selected from cancer, inflammatory diseases, diseases of the immune system, diseases of the nervous system, diseases of the circulatory systems, metabolic disorders, pain, diabetes, infections and combinations thereof.
45. The composition for use according to numbered paragraph 44, wherein the infection is a bacterial infection, a fungal infection and/or a parasitic infection.
46. Use of two or more hydroxylhydrocarbons to stabilize a pharmacologically active protein and/or peptide, wherein the two or more hydroxylhydrocarbons comprise i) ethanol and glycerol or ii) ethanol and mannitol.
47. A kit comprising a composition of any one of numbered paragraphs 1 to 20 or numbered paragraph 39.
48. A kit according to numbered paragraph 47, wherein the individual components are separated by a boundary to prevent mixing during storage.
49. The kit according to numbered paragraph 47 or numbered paragraph 48, wherein the pharmacologically active protein and/or peptide may be stored under refrigeration between about 2 and about 8° C. for up to 5 years before use.

### EXAMPLES

The present invention is now further illustrated by means of the following non-limiting examples.

### Sample Preparation

In the examples cited herein, vancomycin was used as the pharmaceutically active peptide. Several compositions were screened, as shown below.

Samples were prepared by mixing the active pharmaceutical ingredient (API), vancomycin hydrochloride, with the additional inactive ingredients followed by the addition of water-for-injection and mixing to form a solution. The solution was filled into rubber-stoppered, crimped-capped, clear-glass vials of suitable size. Specifically, about 0.50 mL of each formulation was filled into 1 mL Type 1 clear glass vials and sealed. The composition was analyzed immediately after manufacturing for the time to assay to establish an initial value for the purity of vancomycin B in the finished product and for comparison with subsequent analytical test results to assess the loss of purity, which were carried out at various subsequent time points. The rest of the stoppered and sealed vials were stored at the desired condition of stress; usually at an elevated temperature to accelerate the degradation of the active ingredient. After, fixed intervals, the vial contents were analyzed by HPLC for assay and related substances (RS).

### Monitoring the Stability of Vancomycin

The stability of vancomycin in any of the examples cited in this invention can be monitored by tracking the content of vancomycin using a reliable and consistent assay method that is known to those trained in the art of formulation development. In the case of this invention, a reverse-phase high performance liquid chromatographic (HPLC) method for assay of vancomycin and related substances described in the British Pharmacopeia (BP) (British Pharmacopoeia 2012, Vancomycin Intravenous Infusion, British Pharmacopoeia Vol III, TSO, Norwich, United Kingdom) was adopted to screen the stability of vancomycin in various product compositions and develop the novel optimum composition. In addition to the conditions described in the BP, test conditions were standardized for the higher concentrations of vancomycin screened and injection volume, as shown in Table 1.

TABLE 1
HPLC parameters for the assay of vancomycin and related substances

| | |
|---|---|
| Column | Agilent Zorbax SB-C18, 4.6 × 250 mm, 5 um |
| Flow Rate | 1.0 ml/min |
| Detection Wavelength | 280 nm |
| Injection Volume | 20 ul |
| Column Temperature | Ambient |
| Run Time | 35 minutes |
| Diluent | Water, HPLC |
| Buffer (Mobile Phase) | (1:500) TEA:Water, HPLC, pH = 3.2 ± 0.2 |
| Mobile Phase A | Buffer:ACN:THF (92:7:1) |
| Mobile Phase B | Buffer:ACN:THF (70:29:1) |
| Target Concentration | 1.0 mg/ml (both standard and sample) |

### Example 1: Stability as a Function of Temperature

Multiple vancomycin batches were prepared as according to the method described above. Ethanol (30% w/w) was weighed in suitable quantity and added to a known weight of vancomycin hydrochloride bulk drug substance (10% w/w) with mixing, followed by the addition of glycerol (10% w/w) and water (50% w/w). The samples were exposed to different temperatures of storage and analyzed after regular intervals to generate an assayed vancomycin content versus time profile at a given temperature. The overall degradation marked by the decreasing levels of vancomycin in the assay samples over time was found to be linear at all temperatures of storage between 2° C. to 50° C. A pseudo first order degradation rate at a given temperature was determined from the slope of the regression line fitted to three or more points on the profile. Table 2 illustrates the influence of temperature on the degradation rates as determined by HPLC assay for intact vancomycin B in solution.

TABLE 2
Pseudo first order degradation rates of vancomycin in a novel
solution composition at different temperatures of storage.

| | | Degradation Rate of |
|---|---|---|
| Ex. No | Temperature (° C.) | Vancomycin B (% loss/day) |
| 1 | 5 | 0.022 |
| 2 | 40 | 0.749 |
| 3 | 50 | 2.7 |

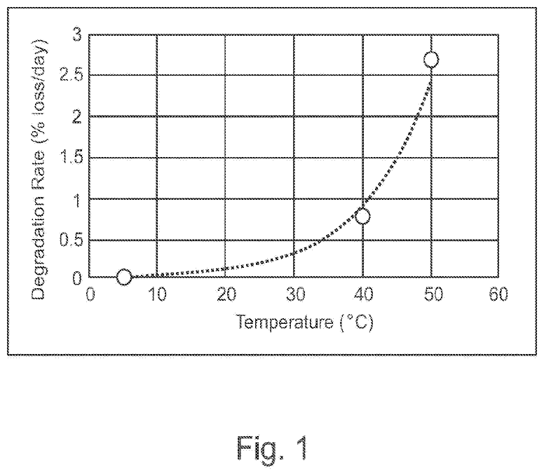

The data from Table 2 is replotted in FIG. 1 to illustrate the exponential increase of degradation rate with increased temperature. This data was used to estimate the degradation rates of vancomycin at a specific temperature within the range of temperatures studied using an Arrhenius-type log-linear plot.

### Example 2: Stability as a Function of the Concentration of API

To establish the effect of the concentration of API in the solution on stability, various solution compositions with a range of conditions were tested and the results were averaged out as shown in Table 3. In this example, all samples were prepared by mixing the API with various types of added excipients, if any, and adjusting the final concentration of vancomycin in solution by adding different quantities of water for injection. The samples were stored at 40° C. and then analyzed over regular intervals by HPLC assay for intact vancomycin in solution to generate a vancomycin purity versus time profile from which the degradation rate was determined as described in Example 1, above. Table 3 illustrates how vancomycin concentration influences its degradation rate regardless of the composition and all other conditions.

TABLE 3
Impact of concentration of vancomycin on its pseudo
first-order degradation rates in solution regardless
of pH and solution compositions tested.

| | Initial Concentration of | Mean Degradation Rate of |
|---|---|---|
| | Vancomycin (% w/w) ± | Vancomycin B (%/day ± |
| Ex. No | Standard Deviation | Standard Deviation) |
| 4 | 0.5 ± 0.0 | 2.13 ± 0.06 |
| 5 | 1.7 ± 0.2 | 2.6 ± 2.14 |
| 6 | 2.0 ± 0.2 | 1.66 ± 1.38 |
| 7 | 9.9 ± 0.1 | 1.02 ± 0.29 |

The results shown in Table 3 demonstrate that the stability of vancomycin tends to improve at higher initial concentrations of vancomycin irrespective of the type and amounts of other inactive ingredients added, and the pH, tonicity, ionic strength, dipole moment, surface tension, viscosity, and other characteristics of the solution phase. The influence of concentration was apparent across all the tested compositions, which ranged from poorly performing to highly optimized. The pronounced impact of concentration on the degradation rate of vancomycin is thought to be due a reduction in the hydrophobic forces due to association of the molecules into dimers and higher order oligomers, which can reduce the overall interfacial area and, hence, the interfacial energy between vancomycin and the solvent, as well as, impart structural rigidity and increase the activation energy needed for the cyclization step in deamidation.

### Example 3: Screening of Excipients

In this example, the vancomycin HCl concentration at the start of the test was adjusted between about 0.5 and 10.0% w/w and the vials were kept at a temperature of 40° C. Various inactive ingredients were added to the solution of vancomycin HCl in water for injection as shown in Table 4 in concentrations ranging from 0.1 to 50%, based on acceptable limits on use and safety as determined from the FDA inactive ingredients database (https://www.fda.gov/Drugs/InformationOnDrugs/ucm080123.htm). Additionally, the solubility, stability and compatibility of each ingredient in the solvent composition were also taken into consideration. The degradation rates as determined from the time versus vancomycin profiles as described in Example 1 and obtained by HPLC assay for pure vancomycin in solution are shown in Table 4.

TABLE 4
Stability of vancomycin in the presence of different
inactive ingredients

| | | Initial | Degradation |
|---|---|---|---|
| | | Concentration | rate |
| Ex. | | of Vancomycin | at 40° C. |
| No | Additional compounds (% w/w) | HCl (% w/w) | (%/day) |
| 8 | — | 2.0 | 4.40 |
| 9 | 4.7% mannitol¹ | 0.5 | 2.11 |
| 10 | 39.9% ethanol | 2.0 | 1.20 |
| 11 | 28.0% ethanol + 6.9% mannitol | 1.4 | 0.81 |
| 12 | 39.2% ethanol + 9.9% glycerol | 2.0 | 0.75 |
| 13 | 30.1% ethanol + 9.9% N-acetyl- | 9.9 | 1.55 |
| | D-alanine | | |
| 14 | 32.2% ethanol | 9.4 | 0.85 |
| 15 | 10.9% glycerol | 9.9 | 1.53 |
| 16 | 28.8% ethanol + 10.6% glycerol | 9.9 | 0.80 |

¹specially coated clear glass vials

As seen from Table 4, the most instability and fastest degradation rate was observed with solutions containing no additional compounds but simply a solution of vancomycin in water for injection, as seen in Ex. No. 8. Each of the examples Ex. No. 9, 10, 14 and 15 comprise a single hydroxylhydrocarbon and demonstrate higher degradation rates compared to Ex. No. 11. Ex. Nos 12 and 16, which comprise two hydroxylhydrocarbons. Ex. No. 13 shows that the addition of N-acetyl-D-alanine to solutions with ethanol increases the degradation rate as seen by comparison with Ex. No. 10. The best degradation rates were observed with compositions comprising ethanol and glycerol (Ex. No 12 and 16) and ethanol and mannitol (Ex. No 11).

### Example 4: Stability as a Function of Concentration of Glycerol

To investigate and optimize the concentration of glycerol in the current invention involving vancomycin, samples according to Example 1 were prepared containing (10% w/w) vancomycin, 30% alcohol 190 Proof by weight (or 28.5% of ethanol by volume) and about 5 to 15% by weight of glycerol as shown in Table 5. The amounts of water for injection were adjusted according to the amount of glycerol added to account for the total weight of each composition such that the concentration of vancomycin and alcohol remained constant. The samples were stored at 50° C. and the degradation rates were determined from the vancomycin purity versus time profiles as described in Example 1 after analyzing at least 3 vials at fixed intervals of storage. At 50° C., typical intervals of storage before the first analysis after time tₒ, was 2 or 3 days, and subsequent analysis after 5-6 days of storage, and after 8-10 days of storage. Table 5 illustrates the influence of glycerol concentration on the degradation rates as determined by HPLC assay for intact vancomycin in solution.

TABLE 5
Stability of vancomycin in the presence of varying
concentrations of glycerol

| | Concentration of | Degradation Rate |
|---|---|---|
| Ex. No | Glycerol (% w/w) | (%/day) at 50° C. |
| 17 | 0 | 2.97 |
| 18 | 5.4 | 2.47 |
| 19 | 10.1 | 2.45 |
| 20 | 15.5 | 2.32 |

As seen in by the results in Table 5, the stability of vancomycin increased with increasing concentrations of glycerol. A similar trend of increasing stability with increasing glycerol concentration was also observed at other temperatures.

### Example 5: Stability of Vancomycin Compositions

Stability of vancomycin in Composition A according to the invention was also tested and compared with the comparative example, Composition B. Composition A comprises approximately 10% (w/w) of vancomycin, 30% (w/w) of 190 proof alcohol, 10% (w/w) glycerol and 50% (w/w) water for injection. Composition B comprises a 10% (w/w) solution of vancomycin in 90% (w/w) water for injection.

Composition A and Composition B were both packaged separately in crimped capped, neutral, Type I clear glass vials and stored at controlled room temperature, between 20 and 25° C. The loss of vancomycin was monitored by HPLC at regular time intervals by monitoring the purity of undegraded vancomycin in the product formulation as shown in Table 6. In Table 6, the purity of vancomycin at various time points is expressed as a % of vancomycin assayed in the corresponding composition immediately after mixing (time tₒ assay) by the HPLC assay method.

TABLE 6
Solution stability of vancomycin in compositions
A and B at 22.5 ± 2.5° C.

| | % Assay | |
|---|---|---|
| Time | Composition A | Composition B |
| T₀ | 100.0 | 100.0 |
| 10 d | 99.4 | — |
| 1 mo | 97.3 | 91.1 |
| 3 mo | 95.7 | 79.3¹ |
| 6 mo | 90.5 | 58.31 |

¹Appearance of white precipitate from the crystalline degradation products of vancomycin

As seen from Table 6, within just three months, Composition B shows significant degradation in comparison to Composition A according to the invention. Composition B also showed precipitation of crystalline degradation products of vancomycin, which was not observed in Composition A for over 6 months.

Further experiments showed that under refrigerated storage, 2-8° C., Composition A according to the invention does not show any measurable degradation even after 6 months.

1. A pharmaceutical composition comprising a cyclicpeptide, a solvent and two or more hydroxylhydrocarbons, wherein the two or more hydroxylhydrocarbons comprise i) ethanol and glycerol; or ii) ethanol and mannitol.

2. The pharmaceutical composition according to claim 1, wherein the cyclicpeptide is selected from actaplanin, anidulafungin, atosiban, bacitracin, carbetocin, caspofungin, calcitonin, calcitonin-salmon, cibacalcin, colistin A and B, complestatin, cyclosporin, dalbavancin, daptomycin, desirudin, desmethylvancomycin, desmopressin, dulaglutide, eptifibatide, insulin, insulin aspart, insulin degludec, insulin detemir, insulin glargine, insulin glulisine, insulin lispro, insulin recombinant, lepirudin, linaclotide, lysine vasopressin, micafungin, murepavadin, nesiritide, oritavancin, oxytocin, plecanatide, pramlintide, ristocetin, teicoplanin, telavancin, vancomycin, vasopressin, ziconotide, β-avoparcin or pharmaceutical salts thereof, stereoisomers thereof, tautomers thereof, or derivatives thereof.

3. The pharmaceutical composition according to claim 1, wherein the cyclicpeptide is present in an amount of about 0.01 to about 50 weight percent based on the total weight of the composition.

4. The pharmaceutical composition according to claim 1, wherein the composition is a solution, a suspension, or a mixture thereof.

5. The pharmaceutical composition according to claim 1, wherein the solvent is a polar solvent, selected from water, dimethylsulfoxide, dimethylacetamide, monoethanolamine, polyethylene glycol, polypropylene glycol and mixtures thereof.

6. The pharmaceutical composition according to claim 1, further comprising sweeteners selected from glucose, sucrose, sorbitol, xylitol, maltitol, sucralose, sodium saccharin, aspartame, stevia or another glycoside and mixtures thereof.

7. The pharmaceutical composition according to claim 1, wherein the total amount of hydroxylhydrocarbon is from about 1 to about 60 weight percent based on the total weight of the composition.

8. The pharmaceutical composition according to claim 1, wherein the amount of ethanol is from about 1 to about 40 weight percent based on the total weight of the composition.

9. The pharmaceutical composition according to claim 1, wherein the amount of glycerol or mannitol is from about 1 to about 22 weight percent based on the total weight of the composition.

10. The pharmaceutical composition according to claim 1, wherein at least 90% by weight of the cyclicpeptide is present in the composition after being stored for 30 days at between about 20 and about 25° C., based on the initial amount of the cyclicpeptide.

11. The pharmaceutical composition according to claim 1, wherein at least 97% by weight of the cyclicpeptide is present in the composition after being stored for 30 days at between about 2 about and 8° C. based on the initial amount of the cyclicpeptide.

12. The pharmaceutical composition according to claim 1, wherein the loss of purity of the cyclicpeptide when stored between about 20 and about 25° C. is no more than 0.3% per day.

13. The pharmaceutical composition according to claim 1, wherein the loss of purity of the cyclicpeptide when stored between about 2 and about 8° C. is no more than 0.03% per day.

14. A method of treating a diseased condition in a subject in need thereof comprising administering to the subject a pharmaceutically effective amount of the pharmaceutical composition according to claim 1.

15. The method according to claim 14, wherein the diseased condition is selected from cancer, inflammatory diseases, diseases of the immune system, diseases of the nervous system, diseases of the circulatory systems, metabolic disorders, pain, diabetes, infections, such as a bacterial infection, a fungal infection and/or a parasitic infection, and combinations thereof.
